(19) United States
(12) Patent Application Publication
(10) Pub. No.: US 2018/0103849 A1
(43) Pub. Date: Apr. 19, 2018

(54) OBJECT INFORMATION ACQUIRING APPARATUS AND SIGNAL PROCESSING METHOD

(71) Applicant: CANON KABUSHIKI KAISHA, Tokyo (JP)

(72) Inventors: Naoya Iizuka, Yokohama-shi (JP); Kenichi Nagae, Yokohama-shi (JP)

(21) Appl. No.: 15/726,668

(22) Filed: Oct. 6, 2017

(30) Foreign Application Priority Data
Oct. 14, 2016 (JP) 2016-202182

(51) Int. Cl. A61B 5/00; A61B 8/00

(52) U.S. Cl. CPC A61B 5/0095; A61B 5/0033; A61B 8/4272; A61B 8/44; A61B 5/0073

(57) ABSTRACT

The present invention provides an object information acquiring apparatus including an irradiating unit, receiving elements which receive acoustic waves generated from an object and output electric signals, a probe which has a supporting member and on which the receiving elements are arranged in the supporting member such that respective directivity axes are converged, a weighting processing unit which performs weighting on electric signals. The weighting processing unit applies higher weights to a first electric signal corresponding to higher intensity of the acoustic wave.

### BACKGROUND OF THE INVENTION

### Field of the Invention

The present invention relates to an object information acquiring apparatus and to a signal processing method.

### Description of the Related Art

In a photoacoustic imaging technique, which is an imaging technique using light, an object is first irradiated with pulsed light generated from a light source. When the irradiating light is propagated/diffused in the object, the energy of the light is absorbed by a plurality of inner portions of the object to generate acoustic waves (hereinafter referred to as photoacoustic waves). By receiving the photoacoustic waves using a probe (transducers) and performing analytical processing of reception signals in a processing device, information related to the characteristic values of the inner portions of the object is acquired as image data. This allows the distribution of the characteristic values inside the object to be visualized.

In recent years, a preclinical study which acquires an image of a blood vessel from a small animal using photoacoustic imaging and a clinical study which applies photoacoustic imaging to a diagnosis of breast cancer or the like have actively been pursued. U.S. Pat. No. 6,216,025 discloses a technique which uses a probe in which a plurality of receiving elements that detect photoacoustic waves are disposed at different positions along a generally hemispherical surface to visualize the characteristic values of the inner portions of an object. This technique orients the directions in which the plurality of receiving elements disposed over the generally spherical surface have high reception directivities to a predetermined region to allow the predetermined region to be visualized with high resolution or with low noise and high sensitivity.
Patent Literature 1: U.S. Pat. No. 6,216,025

### SUMMARY OF THE INVENTION

A photoacoustic wave generated from an absorber having a cylindrical shape, such as a blood vessel, has a high directivity. Accordingly, the photoacoustic wave propagated from the absorber having the cylindrical shape is received by those of the plurality of receiving elements included in the probe which are located within a limited region. On the other hand, image reconstruction is performed using signals from all the receiving elements included in the probe so that reception signals not derived from the photoacoustic waves are also used for the reconstruction. As a result, a problem arises in that averaging reduces the intensity of an image of a blood vessel to be visualized.

Particularly when the receiving elements are arranged in a region smaller than the hemisphere, i.e., when the probe has a limited field of vision, a problem arises in that a blood vessel extending in a direction which is more nearly perpendicular to the aperture plane of the probe is more susceptible to the influence of the limited field of vision and the image intensity decreases.

The present invention has been achieved in view of the foregoing problems. An object of the present invention is to allow an absorber which generates a photoacoustic wave having a directivity to be excellently imaged.

The present invention provides an object information acquiring apparatus, comprising:

an irradiating unit which emits light;

a plurality of receiving elements which receive acoustic waves generated when an object is irradiated with the light, convert the acoustic waves to electric signals, and output the electric signals;

a probe which has a supporting member and on which the plurality of receiving elements are arranged in the supporting member such that respective directivity axes of the receiving elements are converged;

a weighting processing unit which performs weighting on a plurality of electric signals output from the plurality of receiving elements; and

an information acquiring unit which acquires characteristic information of the object using the electric signals, wherein

the plurality of receiving elements include a first receiving element and a second element, and

the weighting processing unit applies weights to the electric signals such that a weight applied to a first electric signal corresponding to the first receiving element receiving a first acoustic wave of first intensity is higher than a weight applied to a second electric signal corresponding to the second receiving element receiving a second acoustic wave of second intensity lower than the first intensity.

The present invention also provides a signal processing method for processing electric signals generated through reception and conversion, by a plurality of receiving elements, of acoustic waves generated upon irradiation of an object with light, the plurality of receiving elements being arranged on a supporting member such that respective directivity axes of the receiving elements are converged, the method comprising the steps of:

performing weighting on a plurality of electric signals output from the plurality of receiving elements; and

acquiring characteristic information of the object using the electric signals, wherein

the plurality of receiving elements include a first receiving element and a second element, and

in the weighting, a weight applied to a first electric signal corresponding to the first receiving element receiving a first acoustic wave of first intensity is higher than a weight applied to a second electric signal corresponding to the second receiving element receiving a second acoustic wave of second intensity lower than the first intensity.

The present invention allows an absorber which generates a photoacoustic wave having a directivity to be excellently imaged.

Further features of the present invention will become apparent from the following description of exemplary embodiments with reference to the attached drawings.

### BRIEF DESCRIPTION OF THE DRAWINGS

FIG. 1 is a schematic diagram showing an overall configuration of a photoacoustic imaging apparatus;

FIGS. 2A to 2F are cross-sectional views showing photoacoustic waves from a cylindrical absorber which correspond to the angles of the cylindrical absorber;

FIGS. 3A to 3F are plan views showing photoacoustic waves from the cylindrical absorber which correspond to the angles of the cylindrical absorber;

FIGS. 4A to 4C are views showing reconstruction images of the cylindrical absorber which correspond to the angles of the cylindrical absorber;

FIG. 5 is a flowchart illustrating photoacoustic imaging involving weighting;

FIG. 6 is a view showing a screen displayed when a weight pattern is specified;

FIGS. 7A to 7C are views showing an example of the weight pattern;

FIG. 8 is a view showing an image displayed on a display unit;

FIGS. 9A and 9B are views showing a weight pattern in a second embodiment;

FIGS. 10A to 10C are views showing reconstruction images in the second embodiment;

FIGS. 11A and 11B are views showing a weight pattern in a third embodiment;

FIG. 12 is a view showing an image displayed on the display unit in the third embodiment; and

FIGS. 13A and 13B are views showing weight patterns in a fourth embodiment.

### DESCRIPTION OF THE EMBODIMENTS

Referring to the drawings, the following will describe preferred embodiments of the present invention. However, the dimensions, materials, and shapes of components described below, relative positioning thereof, and the like are to be appropriately changed in accordance with a configuration of an apparatus to which the invention is applied and various conditions and are not intended to limit the scope of the invention to the following description.

The present invention relates to a technique which detects photoacoustic waves propagated from an object to generate and acquire the characteristic information of the inner portions of the object. Accordingly, the present invention is considered to be an object image acquiring apparatus, a control method therefor, an object information acquiring method, or a signal processing method. The present invention is also considered to be a program for causing an information processing device including hardware resources such as a CPU and a memory to implement each of the foregoing methods, or a non-transitory storage medium which can be read by a computer storing the program.

The object information acquiring apparatus of the present invention includes a photoacoustic imaging apparatus using a photoacoustic effect which irradiates an object with light (an electromagnetic wave) to receive acoustic waves generated in the object and acquire the characteristic information of the object as image data. In this case, the characteristic information is information on characteristic values corresponding to a plurality of respective positions in the object which are generated using reception signals derived from the received photoacoustic waves.

The characteristic information acquired by photoacoustic measurement is values reflecting the amount of absorbed light energy and the absorptance thereof. The characteristic information includes, e.g., the source of an acoustic wave generated by irradiation with light at a single wavelength and an initial sound pressure in the object or a light energy absorption density and an absorption coefficient which are derived from the initial sound pressure. From the characteristic information obtained using a plurality of different wavelengths, the concentration of a substance forming a tissue can be acquired. By obtaining an oxygenated hemoglobin concentration and a deoxygenated hemoglobin concentration as the substance concentration, an oxygen saturation distribution can be calculated. As the substance concentration, a glucose concentration, a collagen concentration, a melanin concentration, a volume fraction of fat or water, or the like can also be obtained.

On the basis of characteristic information from each position in the object, a two-dimensional or three-dimensional characteristic information distribution can be obtained. Distribution data can be generated as image data. The characteristic information may also be obtained not as numerical value data, but as distribution information from each position in the object. Specific examples of the distribution information include an initial sound pressure distribution, an energy absorption density distribution, an absorption coefficient distribution, and an oxygen saturation distribution.

The acoustic wave mentioned in the present invention is typically an ultrasound wave and includes an elastic wave referred to as a sound wave or an acoustic wave. An electric signal to which an acoustic wave is converted by a transducer or the like is referred to also as an acoustic signal. However, the ultrasound wave or acoustic wave recited in the present specification is not intended to limit the wavelength of such an elastic wave. The acoustic wave generated by a photoacoustic effect is referred to as a photoacoustic wave or an optical ultrasound wave. An electric signal derived from a photoacoustic wave is referred to also as a photoacoustic signal. Distribution data is referred to also as photoacoustic image data or reconstruction image data.

The following embodiments will describe, as an object information acquiring apparatus, a photoacoustic imaging apparatus which irradiates an object with pulsed light, receives acoustic waves from the object on the basis of a photoacoustic effect, and analyzes the received acoustic waves to acquire the distribution of light absorbers in the object. The photoacoustic imaging apparatus is appropriate for a diagnosis of blood vessel disease, malignancy, or the like for a human or an animal and follow-up observation after chemotherapy. Examples of the object include a part of a living body such as the breast or hand of the object, an animal other than a human such as a mouse, a non-living object, and a phantom.

### First Embodiment

### System Configuration

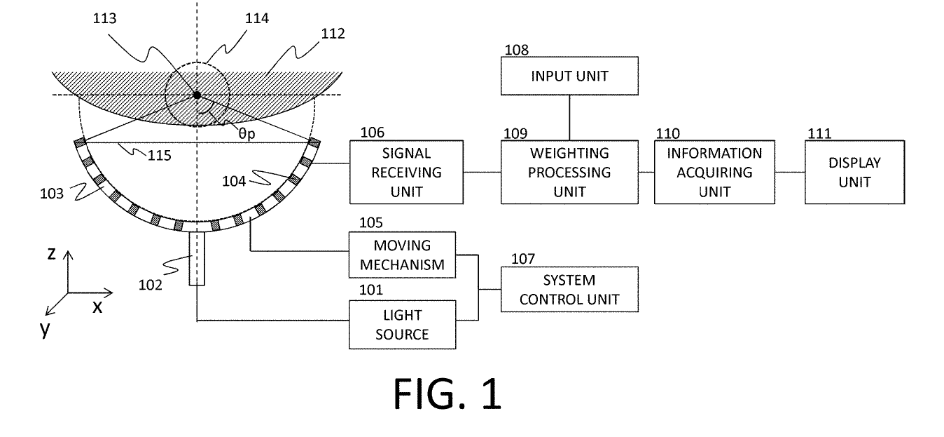

Referring to FIG. 1, a configuration of a photoacoustic imaging apparatus according to the present embodiment will be described. The photoacoustic imaging apparatus according to the present embodiment includes a light source 101, an irradiating unit 102, a probe 103 which receives photoacoustic waves, a plurality of receiving elements 104 included in the probe 103, a moving mechanism 105 which moves the probe 103, a signal receiving unit 106 which processes the reception signals generated by the probe 103, and a system control unit 107 which controls the light source 101 and the moving mechanism 105.

The photoacoustic imaging apparatus further includes an input unit 108 for a user to operate the apparatus, a weighting processing unit 109 which applies weights to the reception signals on the basis of the information received by the input unit, a calculation unit 110 which reconstructs the reception signals into image data, and a display unit 111 which displays generated object information and a user interface (UI) for operating the apparatus.

Note that the calculation unit 110 has a CPU, a main storage device, and an auxiliary storage device. The calculation unit 110 may be included in an independent computer or may also be exclusively designed hardware. Typically, a work station or the like is used as the calculation unit 110. A measurement target is an object 112. The weighting processing unit 109 and the calculation unit 110 may also be configured of common hardware. The calculating unit corresponds to an information acquiring unit in the present invention.

Light Source 101

As the light source 101, a laser light source is desirable to produce a high output. However, instead of a laser, a light emitting diode, a flash lump, or the like can also be used. Various lasers such as a solid-state laser, a gas laser, a dye laser, and a semiconductor laser can be used. A Nd:YAG-excited Ti:sa laser or an alexandrite laser with a high output and a continuously variable wavelength is particularly preferred. By using reception signals derived from light at a plurality of waveforms, a substance concentration can be acquired. Accordingly, a configuration having a plurality of single-wavelength lasers with different wavelengths or a configuration including a variable wavelength laser is preferred.

It is desirable that the wavelength of the pulsed light is a specified wavelength which allows the light to be absorbed in a specified one of the components forming the object and at which the light is propagated into the object. Specifically, when the object is a living body, it is desirable that the wavelength of the pulsed light is at least 700 nm and not more than 1100 nm. To effectively generate photoacoustic waves, the object should be irradiated with the light in a sufficiently short period of time depending on the thermal property of the object. When the object is a living body, the pulse width of the pulsed light (irradiating light) generated from the light source is preferably about 1 nanosecond to 100 nanoseconds.

Irradiating Unit 102

The irradiating unit irradiates the object with the pulsed light emitted from the light source. The irradiating unit includes optical members such as a mirror which reflects the light, a lens which magnifies the light, a diffuser panel which diffuses the light, and a waveguide such has an optical fiber. The irradiating unit leads the irradiating light to the object, while processing the irradiating light such that the distribution of the irradiating light has an intended shape. Note that, in order to improve the safety of the object and widen a diagnosis region, the irradiating light is preferably spread to have a certain area. The irradiating unit of the present embodiment is located in the vicinity of the pole of the probe to irradiate the object with the light from below. However, the positional relationship between the direction in which the object is irradiated and the object is not limited thereto.

Probe 103

The probe 103 has an opening which is upwardly open and a configuration in which the plurality of receiving elements 104 are arranged in the surface (inner circumferential surface) of a hemispherical supporting member around a point 113 as a center of curvature. The plurality of receiving elements 104 are arranged to be widely and generally equally dispersed over the hemispherical surface. The reception sensitivity of each of the receiving elements 104 is highest in a normal direction with respect to the receiving surface thereof for an acoustic wave and has a distribution around the normal direction. The direction in which the reception sensitivity is high is referred to also as a “directivity axis”, which is typically a normal direction with respect to the receiving surface. By converging the respective directivity axes of the receiving elements to the vicinity of the center of curvature of the probe, a high-sensitivity region 114 which is visualized with high sensitivity and high accuracy is formed around the center of curvature 113. As the shape of the probe, not only a hemispherical shape, but also a spherical-crown-like shape, a bowl-like shape, or the like can be used.

The relative positions of the probe 103 and the object vary as the moving mechanism 105 causes the probe 103 to scan the object. As a result of the scanning, the high-sensitivity region 114 is moved relative to the object 112 to allow object information over a wide range to be visualized with high sensitivity and high accuracy. In the present embodiment, the scanning using the probe 103 is performed along an xy-plane. Under the probe 103, the irradiating unit 102 is provided. The light transmitted by an optical transmission system is emitted in a z-direction toward the object 112. The aperture plane of the probe is parallel with the xy-plane.

In the present embodiment, the receiving elements are arranged in the region of the surface of the hemispherical supporting member which is smaller than the hemisphere. That is, the receiving elements are arranged in a region where a polar angle θp in element coordinates around the center of curvature 113 is limited to a degree of less than 90 degrees. This allows an aperture plane 115 of the probe and the object to have a clearance therebetween. As a result, even in the state where the high-sensitivity region 114 is aligned with the deep portion of the object, the probe 103 is allowed to scan over a wide range in the xy-plane. For example, the receiving elements are arranged in the range of the polar angle θp from −70 degrees to +70 degrees.

When the object is under test, an acoustic transmission medium such as water, oil, or gel is injected into the clearance between the probe 103 and the object 112. Alternatively, a thin cup-shaped holding member (not shown) which holds the object 112 may also be used. In this case, the acoustic transmission medium is injected into each of the clearance between the probe 103 and the holding member and the clearance between the holding member and the object 112.

Receiving Elements 104

Each of the receiving elements 104 is a means which detects the acoustic wave generated in the object, converts the detected acoustic wave to an electric signal (photoacoustic signal), and outputs the electric signal. The receiving element 104 is referred to also as an acoustic wave detector or a transducer. As the receiving element 104, a receiving element which can detect an acoustic wave in a frequency band of 100 KHz to 10 MHz generated from a living body is used. Specifically, a transducer using the piezoelectric phenomenon of lead zirconium titanate (PZT) or the like, a transducer using the resonance of light, a transducer using a change in electrostatic capacitance such as CMUT, or the like can be used. As the receiving element 104, a receiving element having a high sensitivity and a wide frequency band is desirable.

As for the receiving elements, the elements can be considered as follows. That is, the plurality of receiving elements include a first receiving element and a second element. And, the weighting processing unit applies weights to the electric signals such that a weight applied to a first electric signal corresponding to the first receiving element receiving a first acoustic wave of first intensity is higher than a weight applied to a second electric signal corresponding to the second receiving element receiving a second acoustic wave of second intensity lower than the first intensity.

Moving Mechanism 105

The moving mechanism 105 moves the probe 103 to change the relative positions of the plurality of receiving elements 104 and the object 112. As the moving mechanism, an automatic stage using a stepping motor or a servo motor can be used. In the present embodiment, the moving mechanism 105 moves the probe 103 along a two-dimensional spiral path. However, the probe 103 may also be moved along a linear or three-dimensional path.

Signal Receiving Unit 106

The signal receiving unit 106 has a means which amplifies the electric signal output from each of the receiving elements and convers the amplified electric signal to a digital signal. Specifically, the signal receiving unit 106 has an amplifier, an A/D converter, a FPGA chip, and the like. The signal receiving unit 106 collects reception signals from the probe 103 at a predetermined sampling rate with a predetermined number of samples in time series and converts the collected reception signals to digital signal data. Since the plurality of reception signals are obtained, it is desirable that a plurality of signals can be simultaneously processed. This can reduce an image formation time. Note that the “reception signal” in the present specification is a concept including an analog signal output from each of the receiving elements as well as a digital signal resulting from the subsequent A/D conversion of the analog signal.

Input Unit 108

The input unit 108 is an input device for performing an image processing operation related to an image such as giving an instruction to set parameters related to the object information to be generated or start measurement or setting observation parameters for the generated object information. In particular, in the present embodiment, the input unit determines a pattern of weights to be applied to the reception signals. The input unit 108 includes a mouse, a keyboard, a touch panel, or the like which receives an input from a user to give an event notification to software such as OS in accordance with an operation by the user. In the case of using the touch panel, the display unit 111 serves also as the input unit 108.

Weighting Processing Unit 109

The weighting processing unit 109 has the function of giving weights in accordance with the weight pattern specified by the input unit to the reception signals. Weight coefficients in the present embodiment are defined in accordance with the z-coordinates of the receiving elements and the reception signals are multiplied by the weight coefficients on a per receiving-element basis. The specific content of weighting processing will be described later. The reception signals multiplied by the weight coefficients in the weighting processing unit are transmitted to the calculating unit. Note that the weighting processing unit 109 also has the function of transmitting the reception signals to the calculation unit 110 without giving the weights thereto (using 1 as a multiplier) for a comparison between weighting effects.

Calculation Unit 110

The calculation unit 110 is a means which processes the signals resulting from digital conversion and reconstructs an image representing information on the optical property or morphology of the inner portion of the object. For the reconstruction, any method may be used such as a Fourier transformation method, a universal back-projection method (UBP method), a filtered back-projection method, or a phasing addition (Delay and Sum) process. The characteristic information of the inner portion of the object is acquired as a set of voxel data when three-dimensional information is acquired. On the other hand, the characteristic information of the inner portion of the object is acquired as a set of pixel data when two-dimensional information is acquired. The generated image is transmitted as photoacoustic image data to the display unit 111 and presented to a user.

Each of the weighting processing unit 109 and the calculation unit 110 can be configured as a program module operated in the same information processing device (such as a PC or work station which has a CPU and a memory and processes information in accordance with a program). However, the weighting processing unit 109 and the calculation unit 110 configured of different information processing devices may also be connected with each other to be used.

Display Unit 111

The display unit 111 displays a photoacoustic image (first image) reconstructed without giving weights to the reception signals from the object, a photoacoustic image (second image) reconstructed by giving weights to the reception signals from the object, and a composite image (third image) of the first and second images. Each of the photoacoustic images is displayed as an arbitrary cross-sectional image or three-dimensional image. The display unit 111 also has a moving image display mode in which photoacoustic images which are sequentially reconstructed every time the probe changes its position are continuously displayed. To ensure a real-time property in a processing flow from the reception of signals to the display of images, it is desirable that image reconstruction processing can be performed at a high speed. As the display unit 111, a fixed-mount-type display such as a liquid crystal display (LCD), a cathode ray tube (CRT), or an organic EL display, a tablet terminal, or the like can be used.

Processing Flow Associated with Reception of Photoacoustic Wave

A description will be given of a processing flow associated with the reception of a photoacoustic wave. In response to an instruction from the system control unit 107, the moving mechanism 105 starts to move such that the probe 103 moves along a predetermined path. The light source 101 generates light at predetermined light emission intervals in response to the instruction from the system control unit 107. The pulsed light generated from the light source 101 at a given time while the probe 103 is moving is propagated by the irradiating unit 102 to irradiate the object 112. A part of the light energy propagated in the object is absorbed by an absorber (such as, e.g., a blood vessel containing a large amount of hemoglobin) which absorbs light at a predetermined wavelength. As a result of thermal expansion of the absorber, a photoacoustic wave is generated.

The probe 103 receives and converts the photoacoustic wave to a time-series reception signal. The reception signals output from the plurality of receiving elements 104 are successively input to the signal receiving unit 106. The signal receiving unit 106 performs the amplification and AD conversion of the reception signals and transmits the digitized reception signals to the weighting processing unit 109.

Directivity of Photoacoustic Wave

The photoacoustic wave generated from an absorber may have a directivity in accordance with the shape of the absorber. For example, the photoacoustic wave generated from a cylindrical absorber has a high directivity in a direction perpendicular to a cylindrical axis. This is because extremely small spherical waves generated from the individual points in the cylindrical absorber are superimposed on each other and consequently an overall wave front is strongly propagated in the direction perpendicular to the cylindrical axis.

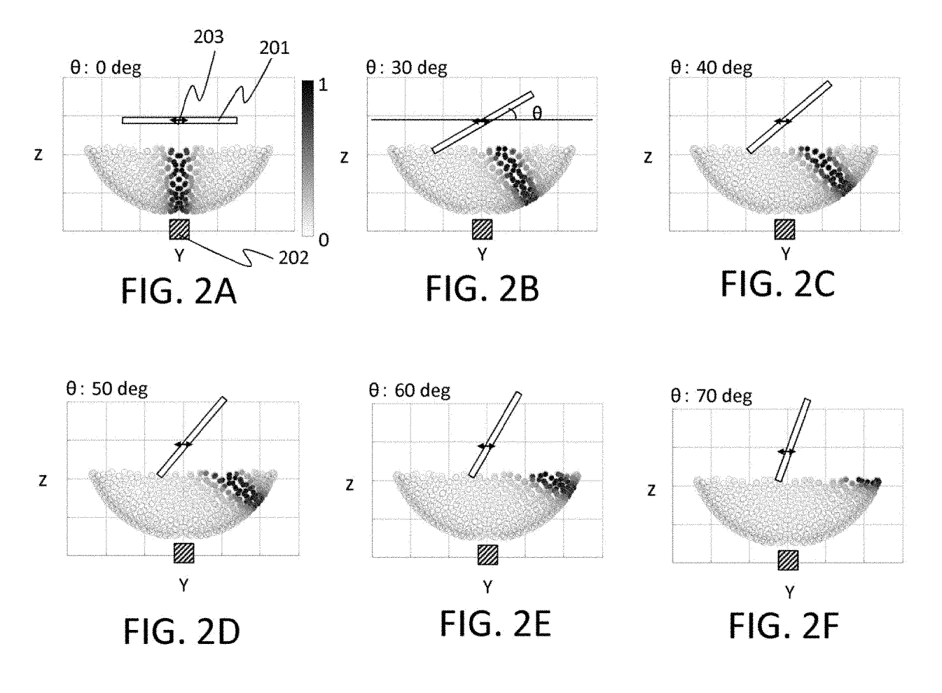

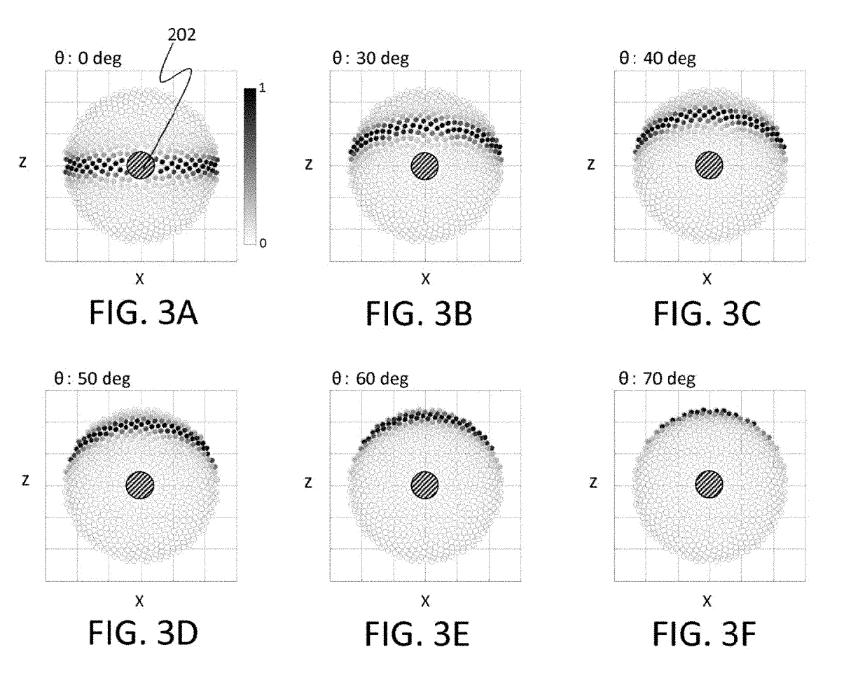

FIGS. 2A to 2F (cross-sectional views) and FIGS. 3A to 3F (plan views) show the intensity distributions of the photoacoustic wave generated from a cylindrical absorber 201 when irradiated with light from a light irradiating unit 202 disposed at the bottom portion of a probe. FIGS. 2A to 2F are different in the angle (inclination angle of the cylindrical absorber) θ formed between the cylindrical absorber 201 and an XY-plane, and FIGS. 3A to 3F are also different in the angle (inclination angle of the cylindrical absorber) θ formed between the cylindrical absorber 201 and the XY-plane. The maximum values of the signals received by individual receiving elements in the probe when the cylindrical absorber 201 is located at the center of curvature of the probe are plotted on a scale normalized between 0 and 1. The positions of the receiving elements in darker colors indicate higher signal intensities.

A double-headed arrow 203 in each of the drawings shows a simulated range of irradiating light at the height of the center of curvature of the probe. As described above, the photoacoustic wave generated from the cylindrical absorber 201 has a high directivity in the direction perpendicular to the cylindrical axis. As a result, it can be seen that the majority of the photoacoustic wave is incident on the belt-like region having a width in accordance with the size of the range of the irradiating light. For example, in each of FIGS. 2A and 3A in which the range of the irradiating light is relatively large, the region on which the photoacoustic wave is incident is relatively large. On the other hand, as the degree of coincidence between the direction of the cylindrical axis and the direction in which the light is incident increases, the range of the irradiating light becomes smaller so that the width of the region on which the photoacoustic wave is incident also becomes smaller.

The present invention aims at improving the intensity of a reconstruction image by performing reconstruction in consideration of an intensity distribution of signals from the individual receiving elements resulting from the directivity of the photoacoustic wave as described above. Specifically, by multiplying the signals from the individual elements by weight coefficients in accordance with the signal intensities, the influence of averaging resulting from the use of the signals from all the elements, including the elements on which the photoacoustic wave is incident in small amounts, is reduced to improve the intensity of the reconstruction image of the absorber.

In image reconstruction processing based on a universal back-projection method or the like, information on a characteristic value such as an initial sound pressure is obtained in accordance with Formula (1).

$$\begin{matrix}[ Math . 1 ] & \\ {p}_{0} ( r ) = {\int }_{{Ω}_{0}} b ( {r}_{0} , t = \vert r - {r}_{0} \vert ) \frac{d {Ω}_{0}}{{Ω}_{0}} & ( 1 )\end{matrix}$$

In Formula (1), p₀(r) represents an initial sound pressure distribution at a position r, b (r₀, t) represents projection data, and dΩ₀ represents the solid angle of a detector with respect to an arbitrary observation point. By back-projecting the projection data in accordance with the integration in Formula (1), the initial sound pressure distribution p₀(r) can be acquired. By integrating intensity data projected by all the individual elements on an arbitrary reconstruction voxel and dividing the resulting value by the sum of the sold angles of the individual elements with respect to the arbitrary observation point, the initial sound pressure corresponding to the arbitrary reconstruction voxel, i.e., the intensity of a reconstruction image is determined.

In the signal processing method of the present invention, such weighting processing as to increase the intensity difference between the receiving element which detects a signal having a relatively high intensity and the receiving element which detects a signal having a relatively low intensity is performed. This can reduce the influence of the averaging and improve the intensity of a reconstruction image of the absorber.

When the cylindrical absorber is inclined relative to the aperture plane of the probe using the center of curvature of the probe as a supporting point, as the inclination angle θ increases, the high-intensity region of the photoacoustic wave moves toward the edge of the probe. When the inclination angle θ exceeds 70 degrees, the number of the elements which receive photoacoustic waves having high intensities decreases, though depending on the shape of the probe or the arrangement of the elements in the probe. This is because, as described above, the receiving elements in the probe are arranged in a range of not larger than the hemisphere and therefore have limited fields of vision. As a result, a phenomenon is observed in which the intensity of a reconstruction image decreases when the inclination angle θ is about 70 degrees or more.

Influence of Directivity on Image and Weighting

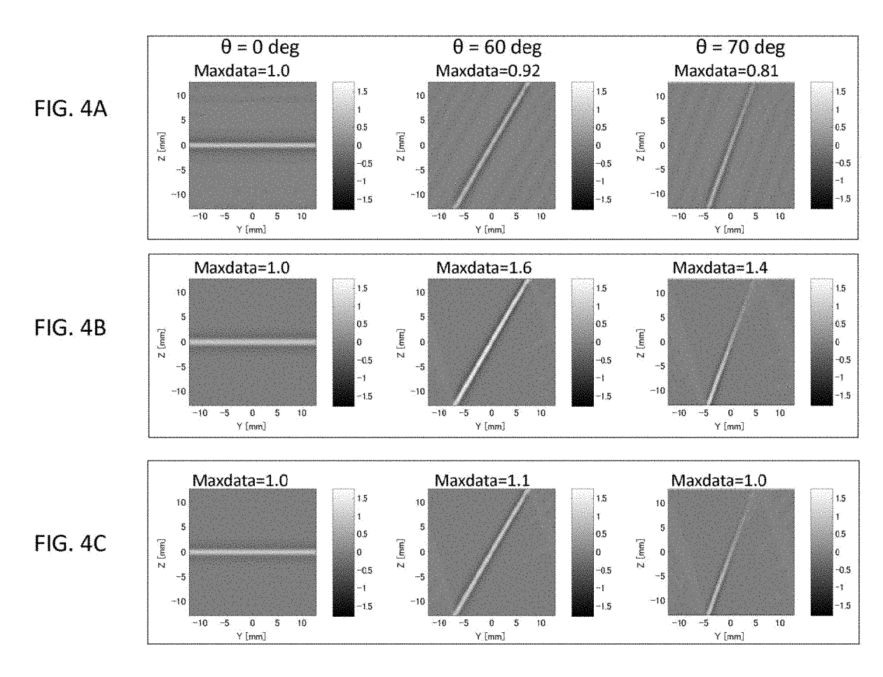

FIG. 4A shows reconstruction images when the cylindrical absorber was disposed to be inclined at angles of 0, 60, 70, and 80 degrees and reconstruction was performed using a conventional method (i.e., method which does not give weights to signals). It can be seen that “Maxdata” showing the maximum intensity in each of the images decreases as the angle increases.

This phenomenon also similarly occurs when a blood vessel in a living body is imaged using a photoacoustic imaging apparatus. A blood vessel at a large inclination angle relative to the aperture plane of the probe, i.e., a blood vessel extending in a depth direction is lower in the intensity of a reconstruction image than a blood vessel at a small inclination angle relative to the aperture plane of the probe, i.e., a blood vessel extending in a horizontal direction. Since a plurality of blood vessels extending in various directions are contained in a living body, a phenomenon is observed in which the S/N ratio of a blood vessel lower in intensity decreases due to the artifact noise of a blood vessel higher in intensity, resulting in low contrast.

Accordingly, in the present invention, reconstruction is performed in which weights in accordance with the signal intensity distribution resulting from the directivities of photoacoustic waves are applied to reception signals. Preferably, such weighting is performed as to increase the contribution of the photoacoustic wave generated from a blood vessel extending in the depth direction in a living body to a reconstruction image and reduce the contribution of the photoacoustic wave generated from a blood vessel extending in a relatively horizontal direction to the reconstruction image. As a result, the image intensity of the blood vessel extending in the depth direction is improved.

FIGS. 4B and 4C show reconstruction images at the individual angles of the absorber when the reception signals are weighted using different weight patterns. In the drawings, all the images are shown in a uniform color scale. In either case, it can be seen that the intensities of the images at the angles of 60 and 70 degrees are higher than the intensities of the normal reconstruction images at the angles of 60 and 70 degrees in FIG. 4A. The details of the individual weight patterns will be described later.

Processing Flow Associated with Weighting of Reception Signals and Image Display

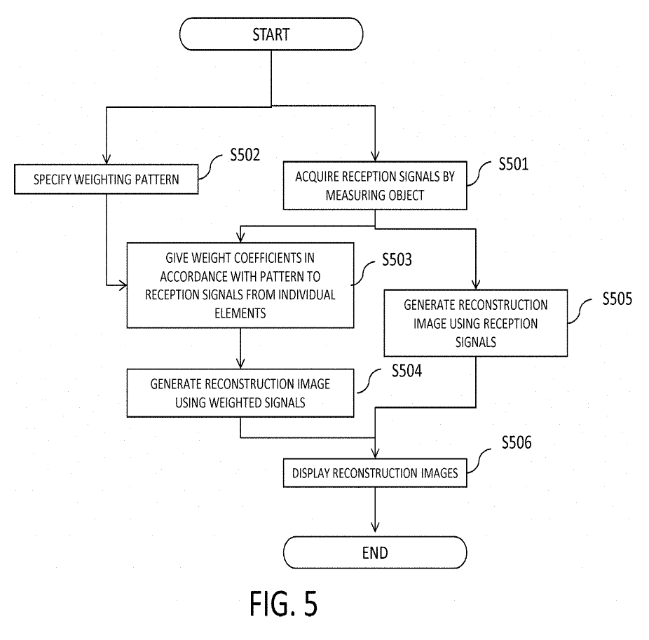

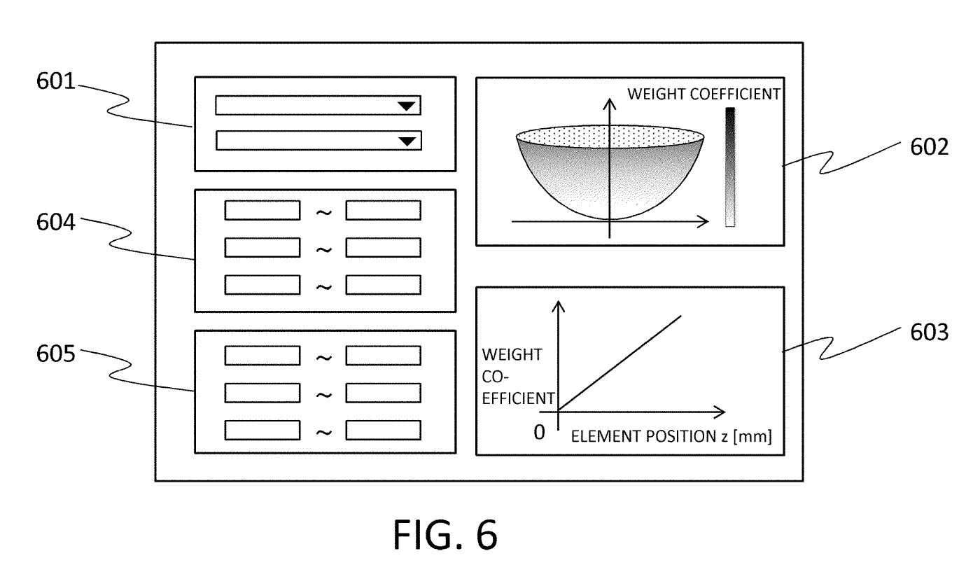

Using FIGS. 5 and 6, the following will describe a flow up to the display of images in the present embodiment. FIG. 5 is a flow chart showing a method of weighting the reception signals and displaying reconstruction images which is implemented by the photoacoustic imaging apparatus according to the present embodiment.

Step S501 is the step of acquiring reception signals derived from the photoacoustic waves generated from the object. First, the plurality of receiving elements receive the photoacoustic waves propagated from the object irradiated with light. Next, the signal receiving unit performs amplification processing and digital conversion processing on the successively input reception signals. The digital reception signals are transmitted to the weighting processing unit.

Note that weighting and image reconstruction need not immediately be performed on the reception signals. It may also be possible to, e.g., store the reception signals in a memory not shown and perform imaging processing on the reception signals later. The present invention can also be considered to be a signal processing apparatus or a signal processing method which perform weighted image reconstruction on the reception signals already stored in the memory. In that case, the signal processing apparatus selects a weight pattern or performs weighted reconstruction processing in accordance with the positions of the elements in the probe, the positional relationships with a light emitting end, or the like stored as the accompanying information of the reception signals.

Step S502 is the step of specifying a pattern of the weights to be applied to the reception signals. The specification of the weight pattern is performed by the user via the input unit. FIG. 6 shows an example of a screen displayed when the weight pattern is specified via the input unit in the present embodiment. In the screen, a weight pattern selecting unit 601, an element arrangement display unit 602, a weight pattern graph display unit 603, an element region specifying unit 604, and a weight coefficient specifying unit 605 are included.

Using the weight pattern selecting unit 601, the user selects the weight pattern to be used among the plurality of weight patterns prepared in advance. Preferably, the weight patterns are categorized according to type and a large item selector and a small item selector are provided to facilitate pattern selection. In accordance with selected pattern, the receiving elements displayed on the element arrangement display unit 602 are displayed in colors corresponding to weight coefficients. In accordance with the selected weight pattern, the weight pattern graph display unit 603 displays a graph showing the weight coefficients with respect to the positions of the receiving elements in the z-direction. The user can recognize the elected weight pattern using the element arrangement display unit 602 and the weight pattern graph display unit 603.

The user can also finely and precisely adjust the values in the weight pattern prepared in advance using the element region specifying unit 604 and the weight coefficient specifying unit 605. When the user finely adjusts the weight pattern, the result thereof is reflected on the element arrangement display unit 602 and the weight pattern graph display unit 603. The weight pattern thus specified is transmitted to the weighting processing unit.

An example is shown herein in which the weight pattern is determined through the specification by the user. However, a configuration may also be used in which the predetermined weight pattern is automatically specified in the weighting processing unit 109 to be used. For example, the pattern may be automatically selected such that a larger weight is applied to the element on which a photoacoustic wave is incident in a larger amount on the basis of the relations among the positions of the individual receiving elements, the position and light irradiation direction of the light irradiating unit, and the position of the object. Alternatively, it may also be possible to determine the weights applied to the individual receiving elements or select the weight pattern on the basis of the intensities of the reception signals from the individual receiving elements.

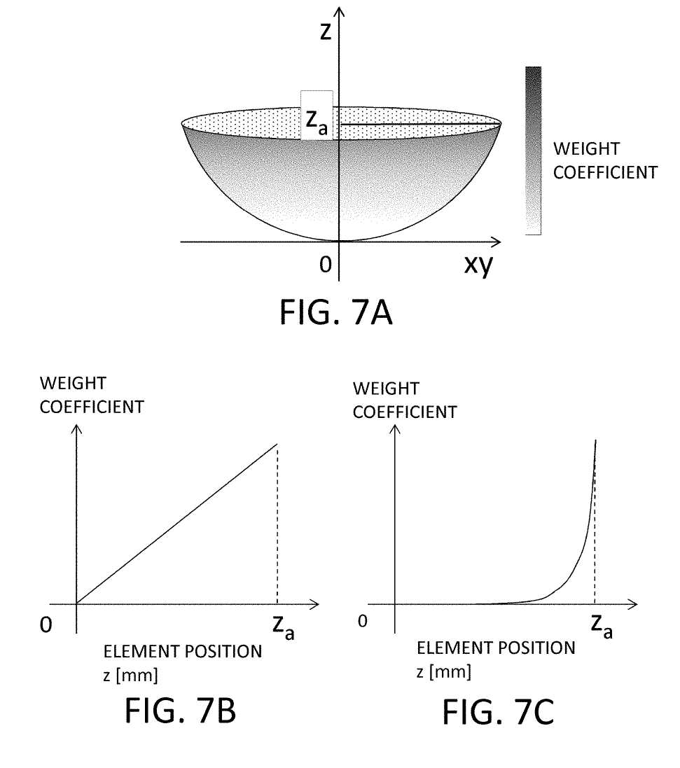

Step S503 is the step in which the weighting processing unit applies the weight pattern to the reception signals. FIGS. 7A to 7C show an example of the weight pattern in the present embodiment. As shown in FIG. 7A, the weight coefficient is determined with respect to each of the coordinates of the positions of the receiving elements in the z-direction. It is assumed herein that z=0 corresponds to the bottom position of the probe and z=za corresponds to the position of the aperture plane of the probe.

FIG. 7B shows the weight coefficient which increases in proportion to the position of the element in the z-direction. By multiplying the reception signal from each of the receiving elements by the weight coefficient, it is possible to image a blood vessel at a large inclination with respect to the aperture plane of the probe with high intensity. The reconstruction image shown in FIG. 4B is generated using this weighting pattern. From the comparison of FIG. 4B with FIG. 4A without weighting, it can be seen that, when the inclination angle of the cylindrical absorber with respect to the aperture plane of the probe is as large as 60 or 70 degrees, an amount of decrease in maximum intensity is reduced and the image of the absorber is clear.

FIG. 7C is a graph when the reciprocal of the relationship between the inclination angle of the cylindrical absorber and the intensity of the reconstruction image is used as the weight coefficient. As shown in FIGS. 4A to 4C, as the inclination angle of the cylindrical absorber increases, the intensity of the reconstruction image decreases. As also shown in FIGS. 2A to 2F and 3A to 3F, as the inclination angle of the cylindrical absorber increases, the high-sensitivity region of the surface of the probe moves in a positive z-axis direction. Accordingly, the use of a weight pattern such as the curve in FIG. 7C compensates for the decrease of the intensity of the reconstruction image corresponding to the inclination angle of the cylindrical absorber. By giving such a weight pattern to the reception signals, images of the cylindrical absorber at all the inclination angles can ideally be displayed with the same intensity. FIG. 4C shows reconstruction images using this pattern. It can be seen that the intensities of the images at the angles 0, 60, and 70 degrees have approximately equal values.

The weight pattern is not limited to the example described above. The weights may be in an arbitrary pattern intended by the user as long as each of the weight is defined with respect to each of the positions of the elements in the z-direction. By giving such weights to the reception signals, it is possible to correct the intensity of a reconstruction image in accordance with the inclination angle of the cylindrical absorber.

As has been described heretofore, in the present step, by multiplying the reception signal from each of the receiving elements by the weight coefficient in accordance with the specified weight pattern, the weight is applied to the reception signal. The reception signal multiplied by the weight coefficient in the weighting processing unit is transmitted to the calculation unit.

Step S504 is the step of reconstructing the weighted reception signals to photoacoustic image data. In this step, the calculation unit converts the digital reception signals resulting from the multiplication by the weight coefficients and received from the weighting processing unit to reconstruction image data showing information on the optical property or morphology of the inner portion of the object and transmits the reconstruction image data to the display unit.

Step S505 is the step of reconstructing the unweighted reception signals to photoacoustic image data. In this step, the digital reception signals received from the signal receiving unit are converted to reconstruction image data showing information on the optical property or morphology of the inner portion of the object.

Step S506 is the step of displaying the photoacoustic image data on the display unit. In this step, the photoacoustic image data as the object information received from the calculation unit is displayed on the display unit.

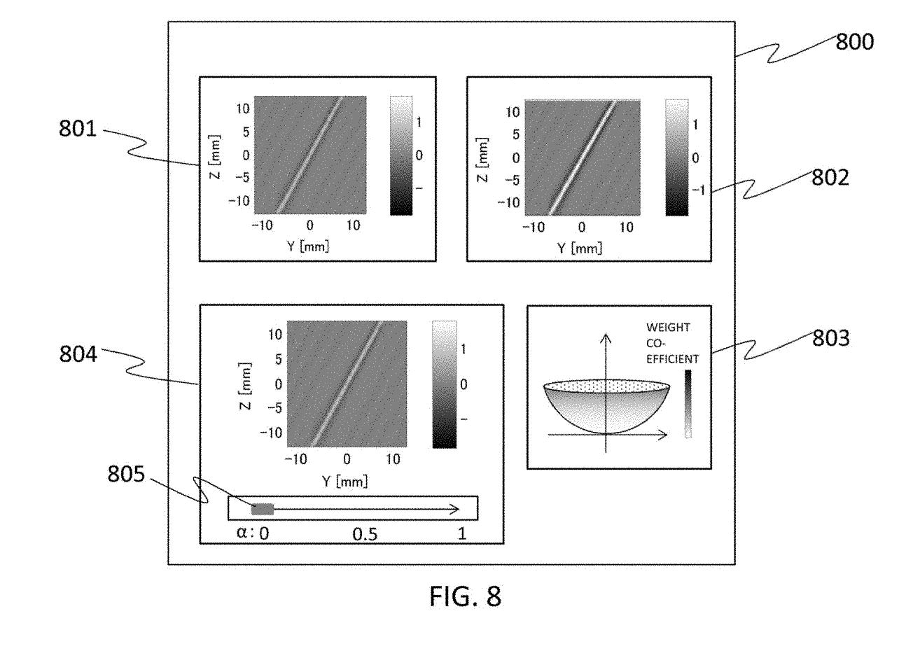

FIG. 8 shows the display screen of the display unit. The display screen 800 includes a normal reconstruction image display unit 801 which displays a normal reconstruction image obtained by reconstructing the reception signals without giving weights thereto, a weighted reconstruction image display unit 802 which displays an image obtained by reconstructing the weighted reception signals, a weight pattern display unit 803 which displays the weight pattern used when the weighted reconstruction image displayed on the weighted reconstruction image display unit 802 was generated, and a composite image display unit 804 which displays an image obtained by summing up the normal reconstruction image and the weighted reconstruction image. The weight pattern displayed on the weight pattern display unit 803 may be a figure representing the respective weight coefficients of the individual receiving elements or a graph of numerical values. The weight pattern display unit 803 may also merely display the name of the weight pattern.

The composite image display unit 804 has a slide bar 805 which can change the ratio α at which the two images are summed up. The summation ratio α is, e.g., a coefficient given by Formula (2) when the image intensity of the composite image at an in-image pixel location (x, y) is I, the image intensity of the normal reconstruction image is Iₙₒᵣₘₐₗ, and the image intensity of the weighted reconstruction image is I_{weight}.

[Math. 2]

I(x,y)=(1−α)*Iₙₒᵣₘₐₗ(x,y)+α*I_{weight}(x,y) (2)

The user can move the slide bar 805 to any position on the UI. For example, when the slide bar 805 is moved to the left end, the summation ratio of the weighted image is 0 so that the normal reconstruction image is displayed on the composite image display unit 804. When the slide bar 805 is moved to the right end, the summation ratio of the normal reconstruction image is 0 so that the weighted reconstruction image is displayed on the composite image display unit 804.

When the signals having the weights which increase as the locations where the photoacoustic waves corresponding to the signals are generated are closer to the edge of the probe (higher positions along the z-axis), as shown in FIG. 7, are reconstructed, a blood vessel at a large inclination angle is imaged with a high intensity, while a blood vessel at a smaller inclination angle is imaged with a low intensity. On the other hand, the unweighted normal reconstruction image has an opposite tendency. Accordingly, by summing up the two reconstruction images, a composite image in which each of the blood vessels at large and small inclinations angles has a high intensity can be obtained.

In the example shown in FIG. 8, the display screen 800 has the normal reconstruction image display unit 801, the weighted reconstruction image display unit 802, the weight pattern display unit 803, and the composite image display unit 804. However, the display method is not limited thereto. It may be possible to display either or only one of the composite image display unit 804 and the weighted reconstruction image display unit 802. Alternatively, it may also be possible to display the normal reconstruction image display unit 801 and either one of the composite image display unit 804 and the weighted reconstruction image display unit 802 in juxtaposition.

As has been described heretofore, the photoacoustic imaging apparatus according to the present embodiment allows an improvement in the image intensity of the absorber as the object to be visualized. By particularly giving the weight coefficients proportional to the z-position of the probe to the reception signals, it is possible to improve the image intensity of a blood vessel extending in the depth direction, which is low in image intensity and less likely to be visualized in conventional reconstruction.

### Second Embodiment

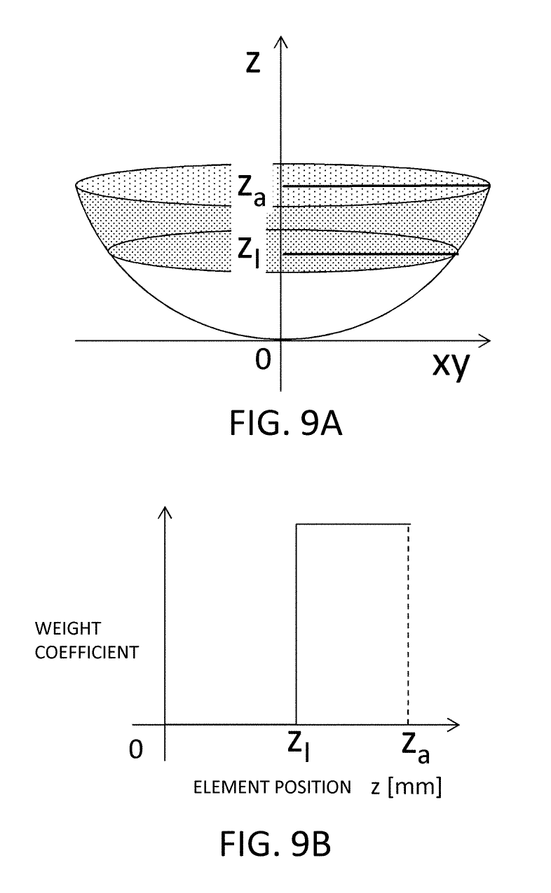

The following will describe a configuration of and processing by a photoacoustic imaging apparatus according to a second embodiment with emphasis on portions different from those in the first embodiment. FIGS. 9A and 9B show a weight pattern in the present embodiment. The weight coefficient is determined with respect to each of the coordinates of the positions of the receiving elements in the z-direction. When an arbitrary height is assumed to be z1, the weight coefficient is 0 in the range given by 0≤z<z1, while the weight coefficient is 1 in the range given by z1≤z≤za, where za represents the height of the aperture plane when z=0 corresponds to the bottom of the probe. That is, the weight pattern is such that, among the plurality of receiving elements included in the probe, only the elements included in the region at a height not lower than z1 are selectively used.

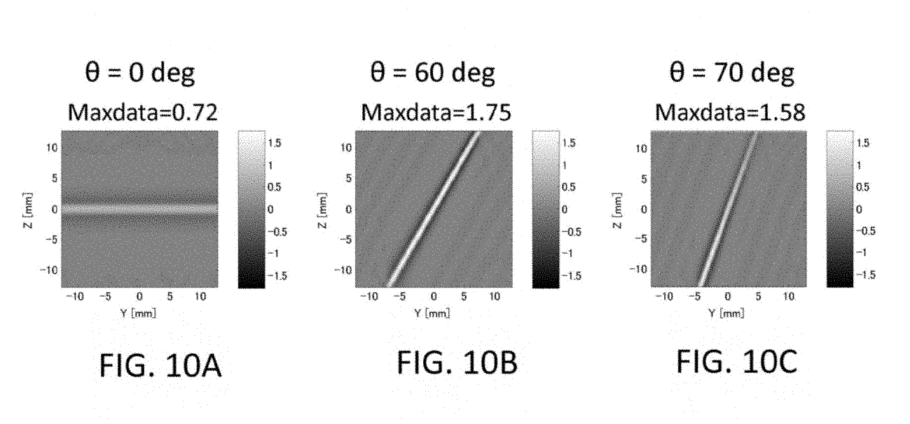

FIGS. 10A to 100 show reconstruction images using the weight pattern in FIGS. 9A and 9B. FIGS. 10A to 100 show the reconstruction images when a cylindrical absorber is disposed to be inclined at angles of 0, 60, and 70 degrees with respect to the aperture plane of the probe. When the maximum value of the normal reconstruction image at an angle of 0 degrees without weighting is assumed to be 1, the respective maximum values of the reconstruction images at the angles of 0, 60, and 70 degrees with weighting are 0.72, 1.75, and 1.58. That is, compared to the case without weighting, the intensity decreases at the angle of 0 degrees, while the intensity increases at each of the angles of 60 and 70 degrees.

As shown in FIG. 4B, the intensities when weights each proportional to the z-position are given are 1.0, 1.6 and 1.4 at the angles of 0, 60, and 70 degrees. Accordingly, it can be said that the weighting in the present embodiment has the higher effect of increasing the image intensity on the cylindrical absorber at a larger angle.

In addition, by giving such a weight pattern as used in the present embodiment to the reception signals, the number of signals used for image reconstruction processing in the calculation unit is reduced to allow a reduction in calculation load. In general, image reconstruction processing involves a large amount of calculation. Particularly when real-time reconstruction is performed following the reception of photoacoustic waves, the ratio of the time period required for the reconstruction processing to the time period from the reception of the photoacoustic waves to the display of a reconstruction image is high. Therefore, it is desirable to minimize the reconstruction processing time. By giving the 0 weight coefficient to each of the receiving elements included in the specified region defined by the positions of the receiving elements in the z-direction, it is possible to simultaneously perform image enhancement in accordance with the angle at which a blood vessel extends and reduce the time required for the image reconstruction processing.

In the example shown in the present embodiment, the weight coefficient is constant in the range given by z1≤z≤za. However, it may also be possible that the weight coefficient is not constant and may vary in accordance with the z-position. Also, the range of z may be any position or any range between 0 and za. Any weight pattern can be used as long as the weight coefficient in the specified region defined by the z-position is 0 and the data amount of the reception signals used for the image reconstruction processing can be reduced.

### Third Embodiment

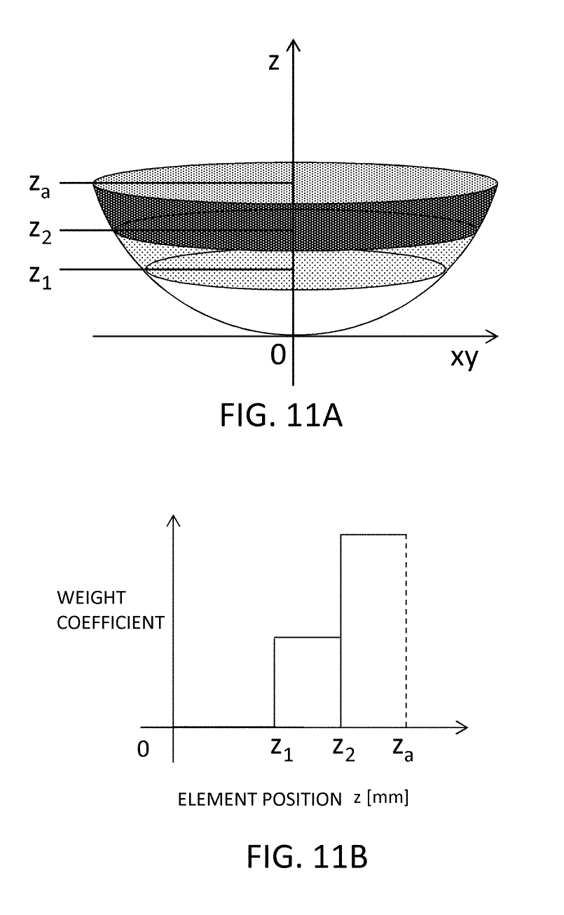

The following will describe a configuration of and processing by a photoacoustic imaging apparatus according to a third embodiment with emphasis on portions different from those in the embodiments described above. FIGS. 11A and 11B show a weight pattern in the present embodiment. The weight coefficient is determined for each of a plurality of regions defined by the positions of the receiving elements in the z-direction. In this example, at arbitrary heights z1 and z2 (z1<z2), the weight coefficient is 0 in the range of a region 1 defined by 0≤z<z1, the weight coefficient is 1 in the range of a region 2 defined by z1≤z≤z2, and the weight coefficient is 2 in the range of a region 3 defined by z2≤z≤za.

Methods for performing the reconstruction processing using the reception signals to which the weight pattern as described above is applied are roughly divided into two categories. A method in the first category does not uses the signals from the receiving elements in the region 1 and applies the respective weight coefficients to the reception signals from the receiving elements included in the regions 2 and 3 to reconstruct an image. A method in the second category does not use the signals from the receiving elements in the region 1 and uses only the reception signals from the receiving elements included in the regions 2 and 3 to generate respective reconstruction images for the individual regions. When image reconstruction is performed on a per region basis using the reception signals from the receiving elements included in each of the regions, since each of the regions is defined by the position thereof in the z-direction, an image in which a blood vessel at an angle corresponding to each of the regions is particularly enhanced is reconstructed.

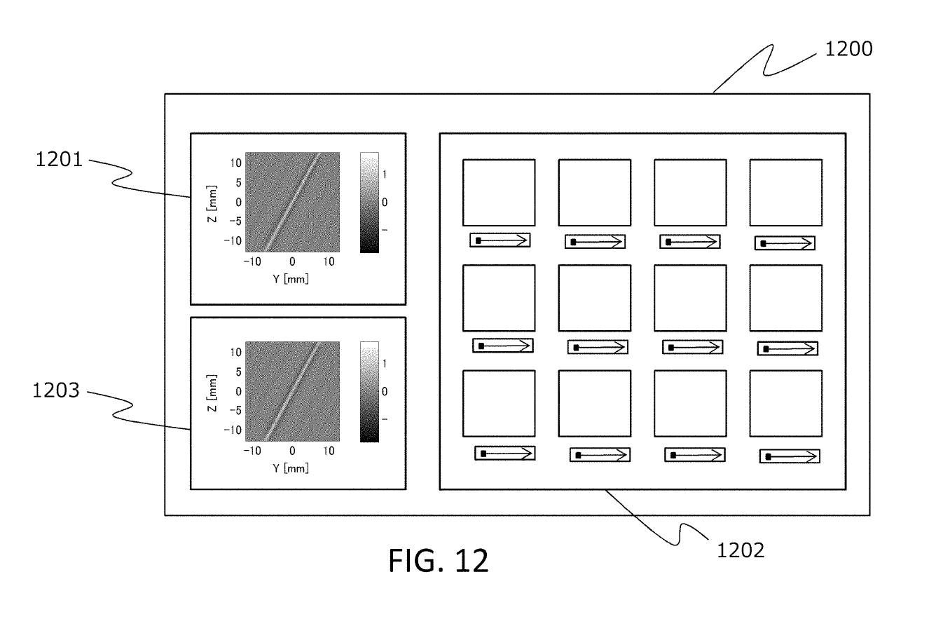

FIG. 12 shows an example of the display unit in the present embodiment. A display screen 1200 includes a normal reconstruction image display unit 1201 which displays a normal reconstruction image obtained by reconstructing the reception signals without giving weights thereto, a weighted reconstruction image display unit 1202 which displays a plurality of images obtained by individually reconstructing the reception signals on a per region basis, and a composite image display unit 1203 which displays an image obtained by summing up the normal reconstruction image and the weighted reconstruction image. The weighted reconstruction image display unit 1202 has a slide bar which can change the ratio at which the reconstruction images corresponding to the individual regions are summed up. By adjusting the slide bar to an arbitrary value, the user allows the composite image display unit 1203 to display the composite image in which a blood vessel extending in an intended angle is enhanced.

### Fourth Embodiment

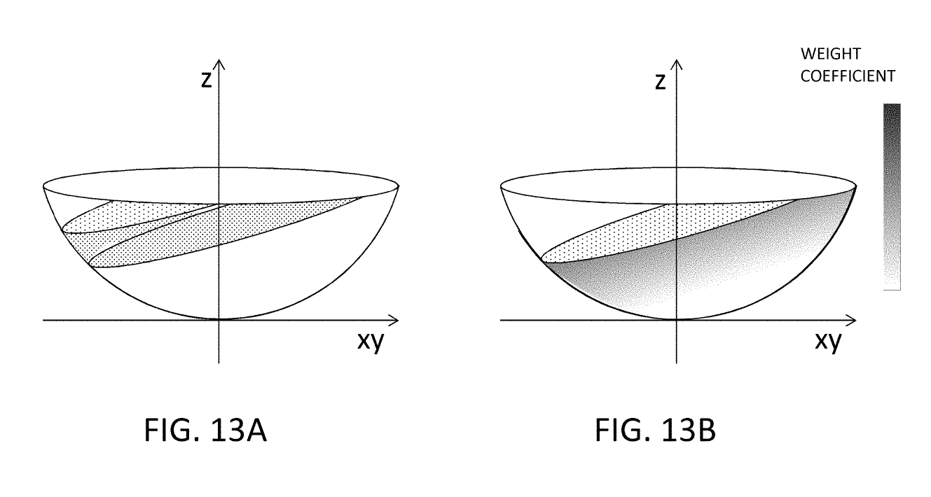

The following will describe a configuration of and processing by a photoacoustic imaging apparatus according to a fourth embodiment with emphasis on portions different from those in the embodiments described above. FIG. 13A shows an example of a weight pattern in the present embodiment. The weight coefficient in the present embodiment is applied to the reception signal from each of the receiving elements included in an arbitrary belt-like region of the surface of the probe. That is, when the probe has a hemispherical or spherical-crown-like shape, the same weight is applied to each of the plurality of receiving elements included in the region having a generally spherical belt-like shape.

As shown in FIGS. 2A to 2F and 3A to 3F, a projection of a cylindrical wave generated from the cylindrical absorber present on the z-axis on the inner surface of a sphere is a belt-like region defined by the region irradiated with light and the center position and polar angle of the cylindrical absorber. Majority of the photoacoustic waves are incident on the belt-like region. Accordingly, the present embodiment is intended to obtain a high enhancing effect by performing image reconstruction using the reception signals derived from only the receiving elements included in the belt-like region.

However, when the position and angle of a blood vessel as an object to be observed are unknown, it is impossible to determine only one weighted element region in advance. Accordingly, in the present embodiment, belt-like regions in a plurality of patterns are specified and image reconstruction corresponding to the individual regions is performed a plurality of times. The reconstruction images generated for the individual regional patterns may be displayed in juxtaposition in the same manner as in the example shown in the third embodiment or a composite image of the individual reconstruction images may also be displayed. Alternatively, it may also be possible that the user specifies an arbitrary composite coefficient or the calculation unit has a program which produces a composite image selectively from target images having relatively high intensities. Still alternatively, the calculation unit may also have the function of estimating the center position and polar angle of the cylindrical absorber from the intensities of the reconstruction images corresponding to the individual regions.

To reduce the number of patterns, it may also be possible to have a weight pattern having a large region, as shown in FIG. 13B, to allow weighting to be performed in accordance with the weighting direction in the region.

As described above, the photoacoustic imaging apparatus according to the present embodiment allows an improvement in the image intensity of the absorber as an object to be visualized.

Note that, when the photoacoustic wave generated from the absorber has a directivity, the present invention applies weights to the respective electric signals derived from the plurality of receiving elements to thus achieve the effect. Accordingly, the present invention is intended not only for a blood vessel as the absorber. For example, a cylindrical absorber other than a blood vessel, such as a lymph vessel, can also be a measurement target. The present invention is applicable to any case where a photoacoustic wave shows any directivity on the basis of the shape of the absorber other than a cylindrical shape.

While the present invention has been described heretofore with reference to the specific embodiments, it is to be understood that the invention is not limited to the specific embodiments described above. The embodiments can be corrected within the scope not departing from the technical idea of the present invention.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-202182, filed on Oct. 14, 2016, which is hereby incorporated by reference herein in its entirety.

What is claimed is:

1. An object information acquiring apparatus, comprising:
an irradiating unit which emits light;
a plurality of receiving elements which receive acoustic waves generated when an object is irradiated with the light, convert the acoustic waves to electric signals, and output the electric signals;
a probe which has a supporting member and on which the plurality of receiving elements are arranged in the supporting member such that respective directivity axes of the receiving elements are converged;
a weighting processing unit which performs weighting on a plurality of electric signals output from the plurality of receiving elements; and
an information acquiring unit which acquires characteristic information of the object using the electric signals, wherein
the plurality of receiving elements include a first receiving element and a second element, and
the weighting processing unit applies weights to the electric signals such that a weight applied to a first electric signal corresponding to the first receiving element receiving a first acoustic wave of first intensity is higher than a weight applied to a second electric signal corresponding to the second receiving element receiving a second acoustic wave of second intensity lower than the first intensity.

2. The object information acquiring apparatus according to claim 1, wherein the weighting processing unit performs the weighting on the basis of respective positions of the object, the plurality of receiving elements, and the irradiating unit and a direction in which the light is emitted.

3. The object information acquiring apparatus according to claim 1, wherein the weighting processing unit performs the weighting on the basis of a directivity of each of the acoustic waves generated from an absorber included in the object.

4. The object information acquiring apparatus according to claim 1, wherein
in a case the object includes a cylindrical absorber, the weighting processing unit applies a larger weight to the electric signal corresponding to the receiving element which is present in a direction perpendicular to a cylindrical axis of the cylindrical absorber among the plurality of receiving elements.

5. The object information acquiring apparatus according to claim 1, wherein the weighting processing unit applies a larger weight to the electric signal corresponding to the receiving element which is included in a belt-like inner circumferential region of the supporting member among the plurality of receiving elements.

6. The object information acquiring apparatus according to claim 1, wherein
in a case that the supporting member has an opening which is upwardly open, and
that the irradiating unit irradiates the object with the light from below, the weighting processing unit applies a larger weight to the electric signal corresponding to the receiving element which is closer to the opening among the plurality of receiving elements.

7. The object information acquiring apparatus according to claim 6, wherein the weighting processing unit distributes the plurality of receiving elements to a plurality of regions on the basis of positions thereof in a z-axis direction, and applies a larger weight to the electric signal corresponding to the receiving element included in the region closer to the opening.

8. The object information acquiring apparatus according to claim 7, wherein the information acquiring unit applies the weights to the electric signals corresponding to the receiving elements included in the plurality of regions, and then performs reconstruction using the plurality of electric signals to acquire the characteristic information of the object.

9. The object information acquiring apparatus according to claim 7, wherein the information acquiring unit individually performs reconstruction for each of the regions to acquire the characteristic information.

10. The object information acquiring apparatus according to claim 1, further comprising an input unit which receives an input from a user, wherein
the weighting processing unit performs the weighting on the basis of the input from the user.

11. The object information acquiring apparatus according to claim 10, wherein the weighting processing unit determines a weight pattern, which is a pattern of the respective weights applied to the plurality of electric signals, on the basis of the input from the user.

12. The object information acquiring apparatus according to claim 1, wherein the weighting processing unit determines a weight pattern, which is a pattern of the respective weights applied to the plurality of electric signals, on the basis of respective positions of the object, the plurality of receiving elements, and the irradiating unit and a direction in which the light is emitted.

13. The object information acquiring apparatus according to claim 1, wherein the information acquiring unit generates a first reconstruction image by performing reconstruction using the electric signals not subjected to the weighting, generates a second reconstruction image by performing reconstruction using the electric signals subjected to the weighting, and generates a composite image of the first and second reconstruction images,
the object information acquiring apparatus further comprising a display unit which displays at least the composite image.

14. The object information acquiring apparatus according to claim 13, further comprising an input unit which receives an input from a user, wherein
the information acquiring unit changes a ratio between the first and second reconstruction images when the composite image is generated on the basis of the input from the user.

15. A signal processing method for processing electric signals generated through reception and conversion, by a plurality of receiving elements, of acoustic waves generated upon irradiation of an object with light, the plurality of receiving elements being arranged on a supporting member such that respective directivity axes of the receiving elements are converged, the method comprising the steps of:
performing weighting on a plurality of electric signals output from the plurality of receiving elements; and
acquiring characteristic information of the object using the electric signals, wherein
the plurality of receiving elements include a first receiving element and a second element, and
in the weighting, a weight applied to a first electric signal corresponding to the first receiving element receiving a first acoustic wave of first intensity is higher than a weight applied to a second electric signal corresponding to the second receiving element receiving a second acoustic wave of second intensity lower than the first intensity.

16. The signal processing method according to claim 15, wherein the step of performing weighting performs the weighting on the basis of respective positions of the object, the plurality of receiving elements, and an irradiating unit which emits light, and a direction in which the light is emitted.

17. The signal processing method according to claim 15, wherein the step of performing weighting performs the weighting on the basis of a directivity of each of the acoustic waves generated from an absorber included in the object.

18. The signal processing method according to claim 15, wherein
in a case the object includes a cylindrical absorber, the step of performing weighting applies a larger weight to the electric signal corresponding to the receiving element which is present in a direction perpendicular to a cylindrical axis of the cylindrical absorber among the plurality of receiving elements.

19. The signal processing method according to claim 15, wherein the step of performing weighting applies a larger weight to the electric signal corresponding to the receiving element which is included in a belt-like inner circumferential region of the supporting member among the plurality of receiving elements.

20. The signal processing method according to claim 15, wherein
in a case that the supporting member has an opening which is upwardly open, and that an irradiating unit for emitting light irradiates the object with the light from below,
the step of performing weighting applies a larger weight to the electric signal corresponding to the receiving element which is closer to the opening among the plurality of receiving elements.

21. The signal processing method according to claim 20, wherein the step of performing weighting distributes the plurality of receiving elements to a plurality of regions on the basis of positions thereof in a z-axis direction, and applies a larger weight to the electric signal corresponding to the receiving element included in the region closer to the opening.

22. The signal processing method according to claim 21, wherein the step of acquiring characteristic information applies the weights to the electric signals corresponding to the receiving elements included in the plurality of regions, and then performs reconstruction using the plurality of electric signals to acquire the characteristic information of the object.

23. The signal processing method according to claim 21, wherein the step of acquiring characteristic information individually performs reconstruction for each of the regions to acquire the characteristic information.

24. The signal processing method according to claim 15, wherein the step of performing weighting determines a weight pattern, which is a pattern of the respective weights applied to the plurality of electric signals, on the basis of respective positions of the object, the plurality of receiving elements, and the irradiating unit and a direction in which the light is emitted.
